(19) United States
(12) Patent Application Publication
(10) Pub. No.: US 2023/0265511 A1
(43) Pub. Date: Aug. 24, 2023

(54) MOLECULAR SIGNATURE OF A COMMON ALOPECIC STATE, ASSOCIATED WITH CELL JUNCTIONS

(71) Applicant: L'OREAL, Paris (FR)

(72) Inventors: Yann MAHE, AULNAY-SOUS-BOIS (FR); Khalid BAKKAR, AULNAY-SOUS-BOIS (FR); Elias BOU SAMRA, AULNAY-SOUS-BOIS (FR)

(21) Appl. No.: 18/002,270

(22) PCT Filed: Jun. 30, 2021
(86) PCT No.: PCT/EP2021/068037

§ 371 Date: Dec. 18, 2022

(30) Foreign Application Priority Data
Jun. 30, 2020 (FR) FR2006900

(51) Int. Cl. C12Q 1/6883

(52) U.S. Cl. CPC C12Q 1/6883; C12Q 1/6869; C12Q 2600/158

(57) ABSTRACT

The present invention relates to a method for the in vitro prognosis and/or diagnosis of a common alopecic state of the scalp in a subject, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

The invention also relates to an in vitro method for evaluating the efficacy of a treatment of a common alopecic state, to a cosmetic treatment method, to the use of a modulator of said abovementioned gene(s), and also to a method for identifying a compound which allows the prevention and/or treatment of a common alopecic state.

The present invention relates to a method for the prognosis and/or diagnosis of a common alopecic state of the scalp.

The human head of hair represents a collection of approximately 150 000 hair strands. Each of those is generated by a specialized appendage of the skin, a truly autonomous organ, the hair follicle. The growth of the hair and its renewal is not a continuous process, and is determined by the activity of the hair follicles and their perifollicular matrix environment. The activity of such follicles is cyclical and comprises essentially four phases. Specifically, the follicle passes successively from a growth phase with hair shaft production (anagen phase) to a rapid involution phase (catagen phase), then to a resting phase with hair loss (telogen phase) which precedes a regeneration phase (neogen phase) so as to again reach the anagen phase. The anagen phase, which is the active or growth phase during which the hair elongates, lasts several years. The very short catagen phase lasts a few weeks. The telogen phase or resting phase lasts a few months. At the end of this resting period, the hair strands fall out and another cycle begins again. The head of hair is thus undergoing constant renewal and, of the approximately 150 000 hair strands which make up a head of hair, approximately 10% are at rest and will be replaced in the months to come.

The natural loss of the hair can be estimated, on average, at a few hundred hairs per day for a normal physiological state. This constant physical renewal process undergoes a natural change during the course of aging: the hairs become finer and their cycles shorter.

Various causes may however bring about substantial temporary or definitive hair loss. Hair loss, in particular common alopecia, for instance androgenetic alopecia, traction alopecia, female pattern hair loss, cicatricial alopecia, chemotherapy-induced or radiotherapy-induced alopecia, telogen effluvium, stress-related alopecia, seasonal alopecia, age-related alopecia and micro-inflammatory alopecia, is essentially caused by disruptions of the hair cycle, as opposed to immune alopecias such as alopecia areata, also known as baldness, alopecia universalis and alopecia totalis. These disruptions firstly cause a shortening of the anagen phase and a gradual thinning of the hair strand, then a decrease in the amount thereof. Gradual miniaturization of the bulbs takes place, in conjunction with isolation of these bulbs by gradual thickening of the collagen matrix of the outer connective sheath. Revascularization around the hair follicle is thus made more difficult cycle after cycle. The hairs regress and become miniaturized until they are no more than an unpigmented down, and this phenomenon leads to gradual thinning of the head of hair.

Certain areas are preferentially affected, in particular the temporal or frontal regions in men, and diffuse alopecia of the crown is instead observed in women.

The term “alopecia” also covers an entire family of hair follicle disorders of which the final consequence is partial or general permanent hair loss. It may more particularly be a case of androgenic alopecia. In a large number of cases, early hair loss occurs in genetically predisposed individuals, which is known as androgenetic alopecia; this form of alopecia most commonly concerns men.

It is known, furthermore, that certain factors, such as hormonal imbalance, physiological stress or malnutrition, can accentuate the phenomenon. In addition, loss or impairment of the hair can be in connection with seasonal phenomena.

In general, any factor that influences these processes, namely the acceleration of the frequency of the cycles, the gradual miniaturization of the bulbs, the gradual thickening of the perifollicular collagen matrix, the thickening of the outer connective sheath, and the decrease in vascularization, will have an effect on the growth of the hair follicles.

It is understood from the aforementioned how important it is to find new biological pathways and new biomarkers for detecting an alopecic state of the scalp, and in particular when said state is not yet visible, in order to be able to reduce and/or slow down hair loss. More particularly, there is a considerable need for biomarkers specific for common alopecia, in particular androgenetic alopecia, which are differentially expressed in the area of growth of the hair strand compared to the area of progression of the alopecia, in order to target it in an adapted manner in a given subject. The aim of the present invention is to satisfy this need.

The applicant has discovered, surprisingly, that the expression of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally CTNNB1 and CTNND2, genes involved in the intercellular junctions of the cells of the scalp and/or of the hair follicle, such as the keratinocytes and fibroblasts, is decreased in the area of progression of the alopecia in a subject with common alopecia.

Thus, the first subject of the present invention is a method for the in vitro prognosis and/or diagnosis of a common alopecic state of the scalp in a subject, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

The present invention also relates to an in vitro method for evaluating the efficacy of a treatment of a common alopecic state, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, before and after treatment, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia..

Another subject of the present invention is a cosmetic treatment method for preventing and/or treating a common alopecic state of the scalp, comprising at least the following steps:
a) measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2;
b) deducing from step a) whether the scalp of said subject exhibits a common alopecic state;
c) if the scalp is identified as exhibiting a common alopecic state in step b), treating said scalp with a cosmetic composition which makes it possible to induce and/or stimulate hair growth and/or to slow down hair loss,
wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

A subject of the present invention is also the use of at least one modulator of the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, or of a cosmetic composition comprising same, for the prevention and/or treatment of a common alopecic state, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia..

Another subject of the present invention relates to a method for identifying a compound which allows the prevention and/or treatment of a common alopecic state, comprising the following steps:
a) bringing a compound to be tested into contact with a biological sample;
b) measuring the expression level, in said biological sample, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2;
c) selecting a compound for which the expression level of said at least one gene measured in step b) is increased compared to its expression level in the absence of said compound,
   wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia..

### Definitions

The abovementioned genes involved in the intercellular junctions of the cells of the scalp can be classified in subfamilies, as presented in table 1 below.

TABLE 1

Genes involved in the intercellular junctions

| | | |
|---|---|---|
| CDH1 | cadherin 1 | Genes involved in the adherent junctions (Simpson C L et al. (2011) Nat Rev Mol Cell Biol 12: 565-80). |
| ACTB | beta-actin | |
| ACTBL2 | beta-actin-like 2 | |
| TUBB | tubulin beta, class I | |
| TUBB2A | tubulin beta 2A | |
| GSN | gelsolin | |
| MYO3B | myosin-IIIb | |
| MYO5B | myosin-Vb | |
| MYO6 | myosin-VI | |
| DSG2 | desmoglein 2 | Genes involved in desmosomes (Simpson C L et al. (2011) Nat Rev Mol Cell Biol 12: 565-80). |
| DSG3 | desmoglein 3 | |
| DSG4 | desmoglein 4 | |
| DSC2 | desmocollin 2 | |
| GJB2 | gap junction protein beta 2 | Genes involved in gap junctions (Simpson C L et al. (2011) Nat Rev Mol Cell Biol 12: 565-80). |
| GJA1 | gap junction protein alpha 1 | |
| GJB6 | gap junction protein beta 6 | |
| GJA3 | gap junction protein alpha 3 | |
| TJP2 | tight junction protein | Genes involved in tight junctions (Simpson C L et al. (2011) Nat Rev Mol Cell Biol 12: 565-80). |
| CLDN8 | claudin 8 | |
| CLDN10 | claudin 10 | |
| CLDN19 | claudin 19 | |
| CTNNB1 | catenin beta 1 | Genes involved in the interaction with CDH1 (Simpson C L et al. (2011) Nat Rev Mol Cell Biol 12: 565-80). |
| CTNND2 | catenin delta 2 | |

The term “common alopecic state” is intended to mean all forms of alopecia except for immune alopecias, and chemotherapy-induced or radiotherapy-induced alopecia. Said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia, preferably the common alopecic state is androgenetic alopecia.

The term “immune alopecia” is intended to mean alopecia of autoimmune origin, chosen from alopecia areata, alopecia universalis and alopecia totalis.

The term “CDH1 gene” is intended here to mean the gene encoding the cadherin 1 protein. The cadherin 1 protein is also known as epithelial cadherin (E-cadherin) or else ovomorulin. This protein is found in the membrane which surrounds the epithelial cells, which are the cells that coat the surfaces and cavities of the body. E-Cadherin belongs to a family of proteins called cadherins, the function of which is to help neighboring cells to adhere to one another (cell adhesion) in order to form organized tissues. It is typically described in Indra et al. (2018) Spatial and temporal organization of cadherin in punctate adherence junctions, Proceedings of the National Academy of Sciences of the United States of America, 115(19):E4406-E4415. The human CDH1 gene sequence is typically referenced under the gene number ID 999 (NCBI). The human CDH1 protein sequence is typically referenced under the UniProt number P12830.

The term “ACTB gene” is intended here to mean the gene encoding the beta-actin protein. It represents one of the six actin isoforms identified in human beings. It is expressed in non-muscle tissues and is a component of the cytoskeleton. It is involved in cell motility, structure and integrity. It is typically described in Erba HP et al. (1988), Molecular and Cellular Biology, Structure, chromosome location, and expression of the human gamma-actin gene: differential evolution, location, and expression of the cytoskeletal beta- and gamma-actin genes, 8(4):1775-1789. The human ACTB gene sequence is typically referenced under the gene number ID 60 (NCBI). The human ACTB protein sequence is typically referenced under the UniProt number P60709.

The term “ACTBL2 gene” is intended here to mean the gene encoding the actin-beta-like 2 protein. It is also known as kappa actin. It is expressed in non-muscle tissues and is a component of the cytoskeleton. It is involved in cell motility, structure and integrity. It is typically described in Saba Ghazanfar et al. (2017), Journal of Proteomics, volume 152, p33-40. The human ACTBL2 gene sequence is typically referenced under the gene number ID 345651 (NCBI). The human ACTBL2 protein sequence is typically referenced under the UniProt number Q562R1.

The term “TUBB gene” is intended here to mean the gene encoding the tubulin beta class I protein. The gene is also known as TUBB5 and the protein is known as tubulin beta 5. This protein forms a dimer with alpha tubulin and acts as a structural component of microtubules. It is typically described in Romina Romaniello et al., European Journal of Medical Genetics, volume 61, p744-754. The human TUBB gene sequence is typically referenced under the gene number ID 203068 (NCBI). The human TUBB protein sequence is typically referenced under the UniProt number P07437.

The term “TUBB2A gene” is intended here to mean the gene encoding the tubulin beta 2A class IIa protein. The protein is also known as tubulin beta 2. This protein forms a dimer with alpha tubulin and acts as a structural component of microtubules. It is typically described in Romina Romaniello et al., European Journal of Medical Genetics, volume 61, p744-754. The human TUBB2A gene sequence is typically referenced under the gene number ID 7280 (NCBI). The human TUBB2A protein sequence is typically referenced under the UniProt number Q13885.

The term “GSN gene” is intended here to mean the gene encoding the gelsolin protein. This cytosolic protein is capable of binding to actin filaments and of creating a local dislocation thereof. It notably promotes vesicle exocytosis through local dissolution of the submembrane cytoskeleton of cells. It is typically described in Eke Gungor H et al. (2016), Allergologia & Immunopathologia, 44(3):221-5. The human GSN gene sequence is typically referenced under the gene number ID 2934 (NCBI). The human GSN protein sequence is typically referenced under the UniProt number P06396.

The term “MYO3B gene” is intended here to mean the gene encoding the myosin-lllb protein. It belongs to the myosin family. Myosins are actin-activated ATPases which move along the actin filaments in the cell. It is typically described in Andrea C.Dose and Beth Burnside (2002), Genomics, 79(5):621-4. The human MYO3B gene sequence is typically referenced under the gene number ID 140469 (NCBI). The human MYO3B protein sequence is typically referenced under the UniProt number Q8WXR4.

The term “MYO5B gene” is intended here to mean the gene encoding the myosin-Vb protein. It belongs to the myosin family. Myosins are actin-activated ATPases which move along the actin filaments in the cell. Myosin Vb helps to determine the position of various components within cells (cell polarity). Myosin Vb also plays a role in the movement of the cell membrane components to the interior of the cell for recycling. It is typically described in Sonal et al. (2014), PLoS Genet. Sep 18;10(9):e1004614. The human MYO5B gene sequence is typically referenced under the gene number ID 4645 (NCBI). The human MYO5B protein sequence is typically referenced under the UniProt number Q9ULV0.

The term “MYO6 gene” is intended here to mean the gene encoding the myosin-VI protein. It belongs to the myosin family. Myosins are actin-activated ATPases which move along the actin filaments in the cell. Myosin VI plays a role in the transport of intracellular vesicles and organelles. It is typically described in Lister Ida et al. (2004), EMBO J. Apr 21;23(8):1729-38. The human MYO6 gene sequence is typically referenced under the gene number ID 4646 (NCBI). The human MYO6 protein sequence is typically referenced under the UniProt number Q9UM54.

The terms “DSG2 gene”, “DSG3 gene” and “DSG4 gene” are intended here to mean the genes encoding respectively the desmoglein 2, desmoglein 3 and desmoglein 4 proteins. They belong to the cadherin cell adhesion molecule family, in particular the desmoglein subfamily. Desmogleins are components of calcium-binding transmembrane glycoproteins of desmosomes, of cell-cell junctions between epithelial and myocardial cell types, and the like. They are typically described in Wu Hong et al. (2003), J Invest Dermatol. Jun;120(6):1052-7 and Masayuki Amagai et al. (2012), J Invest Dermatol. Mar;132(3 Pt 2):776-84. doi: 10.1038/jid.2011.390. The human DSG2 gene sequence is typically referenced under the gene number ID 1829 (NCBI). The human DSG2 protein sequence is typically referenced under the UniProt number Q14126. The human DSG3 gene sequence is typically referenced under the gene number ID 1830 (NCBI). The human DSG3 protein sequence is typically referenced under the UniProt number P32926. The human DSG4 gene sequence is typically referenced under the gene number ID 147409 (NCBI). The human DSG4 protein sequence is typically referenced under the UniProt number Q86SJ6.

The term “DSC2 gene” is intended here to mean the gene encoding the desmocollin 2 protein. Desmocollin 2. This protein is found in numerous tissues, although it seems to be particularly important in cardiac muscle and the skin. Desmocollin 2 is a major component of the specialized structures called desmosomes. These structures help to keep neighboring cells together, thereby conferring strength and stability on the tissues. It is typically described in King Ian et al. (1995), J Invest Dermatol. Sep;105(3):314-21. The human DSC2 gene sequence is typically referenced under the gene number ID 1824 (NCBI). The human DSC2 protein sequence is typically referenced under the UniProt number Q02487.

The term “GJB2 gene” is intended here to mean the gene encoding gap junction protein beta 2. This protein is more commonly known as connexin 26. Connexin 26 is a member of the connexin protein family. Connexin proteins form channels called gap junctions which allow the transport of nutrients, charged atoms (ions) and signaling molecules between adjacent cells. The size of the gap junctions and the types of particles that pass through them are determined by the particular connexin proteins which make up the channel. Gap junctions formed with connexin 26 transport potassium ions and certain small molecules. It is typically described in Iguchi et al. (2003), Experimental dermatology;12(3):283-8. The human GJB2 gene sequence is typically referenced under the gene number ID 2706 (NCBI). The human GJB2 protein sequence is typically referenced under the UniProt number P29033.

The term “GJA1 gene” is intended here to mean the gene encoding gap junction protein alpha 1. This protein is more commonly known as connexin 43. Connexin 43 is one of the 21 connexin proteins. Connexins play a role in cell-to-cell communication by forming channels, or gap junctions, between the cells. Gap junctions allow the transport of nutrients, charged particles (ions) and other small molecules which transport the communication signals necessary between the cells. In addition, connexin 43 attaches (binds) several signaling molecules which can relay the communication signals inside the cell. Connexin 43 is found in numerous tissues, such as the eyes, the skin, the bones, the ears, the heart and the brain, and it plays a role in their development and their function. It is typically described in Salomon D et al. (1994), The Journal of investigative dermatology;103(2):240-7. The human GJA1 gene sequence is typically referenced under the gene number ID 2697 (NCBI). The human GJA1 protein sequence is typically referenced under the UniProt number P17302.

The term “GJB6 gene” is intended here to mean the gene encoding gap junction protein beta 6. This protein is more commonly known as connexin 30. Connexin 30 is a member of the connexin protein family. Connexin proteins form channels called gap junctions which allow the transport of nutrients, charged atoms (ions) and signaling molecules between adjacent cells. The size of the gap junctions and the types of particles that pass through them are determined by the particular connexin proteins which make up the channel. Gap junctions formed with connexin 30 transport potassium ions and certain small molecules. Connexin 30 is present in several different tissues of the body, notably the brain, the inner ear, the skin (in particular the palm of the hands and the sole of the feet), the hair follicles and the nails. It is typically described in Salomon D et al. (1994), The Journal of investigative dermatology;103(2):240-7. The human GJB6 gene sequence is typically referenced under the gene number ID 10804 (NCBI). The human GJB6 protein sequence is typically referenced under the UniProt number 095452.

The term “GJA3 gene” is intended here to mean the gene encoding gap junction protein alpha 3. This protein is more commonly known as connexin 46. Connexin 46 is a member of the connexin protein family. Connexin proteins form channels called gap junctions which allow the transport of nutrients, charged atoms (ions) and signaling molecules between adjacent cells. The size of the gap junctions and the types of particles that pass through them are determined by the particular connexin proteins which make up the channel. It is typically described in Salomon D et al. (1994), The Journal of investigative dermatology;103(2):240-7. The human GJA3 gene sequence is typically referenced under the gene number ID 2700 (NCBI). The human GJA3 protein sequence is typically referenced under the UniProt number Q9Y6H8.

The term “TJP2 gene” is intended here to mean the gene encoding the tight junction protein. This protein is more commonly known as zonula occluden 2, which is a member of the membrane-associated guanylate kinase homolog family. The encoded protein operates as a component of the tight junction barrier in epithelial and endothelial cells and is necessary for correct assembly of the tight junctions. It is typically described in Brandner Johanna et al. (2003), Archives of dermatological research;295(5):211-21. The human TJP2 gene sequence is typically referenced under the gene number ID 9414 (NCBI). The human TJP2 protein sequence is typically referenced under the UniProt number Q9UDY2.

The terms “CLDN8 gene”, “CLDN10 gene” and “CLDN19 gene” are intended here to mean the genes encoding respectively the claudin 8, claudin 10 and claudin 19 proteins. Claudins are integral membrane proteins and components of the strands of tight junctions. Tight junction strands act as a physical barrier to prevent solutes and water from freely passing through the paracellular space between the sheets of epithelial or endothelial cells, and also play essential roles in maintaining cell polarity and signal transductions. They are typically described in Ashikari Daisaku et al. (2017), Cancer Science Jul;108(7):1386-1393, Günzel Dorothee et al. (2009), J Cell Science. May 15;122(Pt 10):1507-17, Konrad Martin et al. (2006), Am J Hum Genet. Nov;79(5):949-57. The human CLDN8 gene sequence is typically referenced under the gene number ID 9073 (NCBI). The human CLDN8 protein sequence is typically referenced under the UniProt number P56748. The human CLDN10 gene sequence is typically referenced under the gene number ID 9071 (NCBI). The human CLDN10 protein sequence is typically referenced under the UniProt number P78369. The human CLDN19 gene sequence is typically referenced under the gene numberlD 149461 (NCBI). The human CLDN19 protein sequence is typically referenced under the UniProt number Q8N6F1.

The term “CTNNB1 gene” is intended here to mean the gene encoding the catenin beta 1 protein.

This protein is present in numerous types of cells and tissues, where it is present mainly at the junctions connecting neighboring cells (adherent junctions). Beta-catenin plays an important role in cell adhesion and in intercellular communication. It is typically described in Hamburg Emily et al. (2012), J Invest Dermatol. Oct;132(10):2469-2472. The human CTNNB1 gene sequence is typically referenced under the gene number ID 1499 (NCBI). The human CTNNB1 protein sequence is typically referenced under the UniProt number P35222.

The term “CTNND2 gene” is intended here to mean the gene encoding the catenin delta 2 protein. This protein is active in the nervous system, where it participates in cell adhesion and plays a role in cell movement. It is typically described in Lu Qun et al. (1999), J Cell Biol. Feb 8;144(3):519-32. The human DSG4 gene sequence is typically referenced under the gene number ID 1501 (NCBI). The human CTNND2 protein sequence is typically referenced under the UniProt number Q9UQB3.

In the context of the invention, the Gene ID (NCBI) and UniProt references cited above are those that were available as of Feb. 27, 2020.

The term “expression level of the gene X” is intended here to mean the mRNA encoded by said gene X or the protein encoded by said gene X. The expression level of the gene X may therefore be measured by quantifying the corresponding mRNA or protein. In one particular embodiment, said expression level of the gene X is the mRNA encoded by said gene X.

Preferably, the expression level corresponds to the concentration or the amount of the expression product (mRNA and/or protein).

The level of the expression product of a gene X may be measured by any technique well known to those skilled in the art. In particular, when the expression product is a protein, the level of the expression product may be measured by means of immunological assays such as ELISA assays, immunofluorescent assays (IFA), radioimmunoassays (RIA), competitive binding assays or Western blots. When the expression product is an mRNA, the level of the expression product can be measured by RT-PCR, qRT-PCR, ddPCR (Droplet Digital PCR), by sequencing, for example by sequencing of NGS type (Next Generation Sequencing) or by ddSEQTM single-cell isolator sequencing.

The expression “method for the in vitro diagnosis of a common alopecic state” denotes here a method for determining whether a subject is suffering from a common alopecic state.

The expression “method for the in vitro prognosis of a common alopecic state” denotes here a method for determining whether there is a risk of a subject suffering from a common alopecic state.

The expression “prevention of a common alopecic state” denotes here a prophylactic or preventive treatment of a common alopecic state, which consists in preventing or delaying the appearance of a common alopecic state, in particular hair loss in the context of androgenetic alopecia.

The expression “treatment of a common alopecic state” denotes here a treatment which consists in reducing, inhibiting or eliminating a common alopecic state, in particular in reducing, inhibiting or eliminating hair loss and/or in promoting hair growth in the context of androgenetic alopecia.

For the purposes of the present invention, the expression “area suspected of being an alopecic area” denotes for example an area of the scalp of a subject which exhibits a loss of hair follicle density, in particular perceived or measured, for example: a sparse head of hair, abnormal hair loss, thinning of the hair.

For the purpose of the present invention, the expression “area suspected of becoming an alopecic area” denotes for example an area of the scalp of a subject having been an alopecic area.

The term “subject” is understood to mean a human being, preferably from 17 to 80 years old, preferentially from 20 to 70 years old, from 23 to 66 years old. The subject is preferably a man.

### DETAILED DESCRIPTION OF THE INVENTION

### Method of Diagnosis

A subject of the present invention is a method for the in vitro prognosis and/or diagnosis of a common alopecic state of the scalp in a subject, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

Advantageously, the biological sample in step a) may be a scalp biopsy and/or a biopsy of one or more hair follicle(s), even better still a biopsy of one or more hair follicle(s) such as at least one follicular transplantation unit, in particular with a diameter of approximately 1 mm.

Preferably, said at least one gene is chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes.

In another preferred embodiment, said at least one gene is chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally chosen from CTNNB1, CTNND2, MYO6, GJB6, CLDN10 and CLDN19.

In this embodiment, said at least one gene is preferably chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes and optionally the group constituted of the CTNNB1, CTNND2, MYO6, GJB6, CLDN10 and CLDN19 genes.

In another embodiment, said at least one gene is chosen from the group constituted of the CDH1, ACTB, ACTBL2, TUBB, TUBB2A, GSN, MYO3B, MYO5B, MYO6 genes and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes.

According to another embodiment, said at least one gene is chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes.

In another embodiment, said at least one gene is chosen from the group constituted of the GJB2, GJA1, GJB6 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes.

According to another embodiment, said at least one gene is chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes.

Regardless of the embodiment, said method of prognosis and/or diagnosis can comprise at least one step a) of measuring the expression level of at least two, three, four or five of the abovementioned genes.

The method according to the invention may also comprise the following steps:
b) comparing the level of said at least one gene measured in step a) with a control;
c) on the basis of the comparison of step b), determining whether the scalp of said subject exhibits a common alopecic state.

The term “comparing” refers to the determining of whether the expression level of said at least one gene is essentially identical to a control or differs from said control. Preferably, the expression level of said at least one gene is considered to be different from a control if the difference observed is statistically significant. If the difference is not statistically significant, the expression level of said at least one gene and the control are essentially identical.

On the basis of this comparison, it is possible to determine whether the scalp of a subject exhibits a common alopecic state.

Thus, said scalp is confirmed as exhibiting a common alopecic state when the expression level of at least one gene chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, is decreased compared to a control level.

Preferably, said scalp is confirmed as exhibiting a common alopecic state when the expression level of at least one gene chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes, is decreased compared to a control level.

In another preferred embodiment, said scalp is confirmed as exhibiting a common alopecic state when the expression level of at least one gene chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally of at least one gene chosen from CTNNB1, CTNND2, MYO6, GJB6, CLDN10, and CLDN19, is decreased compared to a control level.

In this embodiment, said scalp is confirmed as exhibiting a common alopecic state when preferably the expression level of at least one gene chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes, and optionally the group constituted of the CTNNB1, CTNND2, MYO6, GJB6, CLDN10 and CLDN19 genes, is decreased compared to a control level.

In another embodiment, said scalp is confirmed as exhibiting a common alopecic state when the expression level of at least one gene chosen from the group constituted of the CDH1, ACTB, ACTBL2, TUBB, TUBB2A, GSN, MYO3B, MYO5B, MYO6 genes, and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes, is decreased compared to a control level.

According to another embodiment, said scalp is confirmed as exhibiting a common alopecic state when the expression level of at least one gene chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes, is decreased compared to a control level.

In another embodiment, said scalp is confirmed as exhibiting a common alopecic state when the expression level of at least one gene chosen from the group constituted of the GJB2, GJA1 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes, is decreased compared to a control level.

According to another embodiment, said scalp is confirmed as exhibiting a common alopecic state when the expression level of at least one gene chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes, is decreased compared to a control level.

The term “decreased level” is intended here to mean a level that is statistically significantly decreased compared to the control.

In a first embodiment, the “control” or “control level” is an average value determined by measuring the expression level of at least one of said genes in a biological sample from a non-alopecic area in a population of subjects, for example a population of subjects exhibiting a common alopecia, notably androgenetic alopecia.

In a second embodiment, the “control” or “control level” is determined by measuring the expression level of at least one of said genes in a biological sample from a non-alopecic area of the same subject as that of step a).

In vitro method for evaluating the efficacy of a treatment

A subject of the present invention is also an in vitro method for evaluating the efficacy of a treatment of a common alopecic state, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, before and after treatment wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

Preferably, said at least one gene is chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes.

In another preferred embodiment, said at least one gene is chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally chosen from CTNNB1, CTNND2, MYO6, GJB6, CLDN10 and CLDN19.

In this embodiment, said at least one gene is preferably chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes and optionally the group constituted of the CTNNB1, CTNND2, MYO6, GJB6, CLDN10 and CLDN19 genes.

In another embodiment, said at least one gene is chosen from the group constituted of the CDH1, ACTB, ACTBL2, TUBB, TUBB2A, GSN, MYO3B, MYO5B, MYO6 genes and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes.

According to another embodiment, said at least one gene is chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes.

In another embodiment, said at least one gene is chosen from the group constituted of the GJB2, GJA1, GJB6 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes.

According to another embodiment, said at least one gene is chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes.

Regardless of the embodiment, said in vitro method for evaluating the efficacy of the treatment can comprise at least one step a) of measuring the expression level of at least two, three, four or five of the abovementioned genes.

By virtue of the method of evaluation as described, a given treatment will be considered to be efficacious for the treatment of a common alopecic state when the expression level of at least one gene chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, is increased after treatment compared to the expression level of said at least one gene before treatment.

The term “increased level” is intended to mean here a level that is statistically significantly increased compared to the expression level of said at least one gene before treatment.

Preferably, a given treatment will be considered to be efficacious for the treatment of a common alopecic state when the expression level of at least one gene chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes, is increased compared to a control level.

In another preferred embodiment, a given treatment will be considered to be efficacious for the treatment of a common alopecic state when the expression level of at least one gene chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally of at least one gene chosen from CTNNB1, CTNND2, MYO6, GJB6, CLDN10, and CLDN19, is increased compared to a control level.

In this embodiment, a given treatment will be considered to be efficacious for the treatment of a common alopecic state when preferably the expression level of at least one gene chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes, and optionally the group constituted of the CTNNB1, CTNND2, MYO6, GJB6, CLDN10 and CLDN19 genes, is increased compared to a control level.

In another embodiment, a given treatment will be considered to be efficacious for the treatment of a common alopecic state when the expression level of at least one gene chosen from the group constituted of the CDH1, ACTB, ACTBL2, TUBB, TUBB2A, GSN, MYO3B, MYO5B, MYO6 genes, and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes, is increased compared to a control level.

According to another embodiment, a given treatment will be considered to be efficacious for the treatment of a common alopecic state when the expression level of at least one gene chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes, is increased compared to a control level.

In another embodiment, a given treatment will be considered to be efficacious for the treatment of a common alopecic state when the expression level of at least one gene chosen from the group constituted of the GJB2, GJA1 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes, is increased compared to a control level.

According to another embodiment, a given treatment will be considered to be efficacious for the treatment of a common alopecic state when the expression level of at least one gene chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes, is increased compared to a control level.

A treatment will be considered to have no effect if the expression levels of the chosen gene, before and after treatment, are substantially identical, or else if the differences observed are not significant.

When the expression levels of more than one gene are compared, the treatment is considered to be efficacious for the treatment of a common alopecic state, in particular androgenetic alopecia, if, for the majority of the genes tested and preferably for all of the genes tested, taken individually, the treatment is considered to be efficacious. For the other genes chosen, the treatment should preferably have no effect, but must not have the opposite effect.

According to another embodiment, the method for evaluating the efficacy of a treatment of a common alopecic state comprises the diagnosis of a common alopecic state according to the method of the invention, before and after treatment, and the comparison of the expression level differences observed between a biological sample from an area suspected of being an alopecic area or of becoming an alopecic area, and a biological sample from a non-alopecic area.

According to this embodiment of the evaluation of the efficacy of a treatment, the treatment is considered to be efficacious if the difference between the expression levels of the chosen gene(s) in said biological sample from an area suspected of being an alopecic area or of becoming an alopecic area, and said biological sample from a non-alopecic area, is less after treatment compared with what it was before treatment.

When the expression levels of more than one gene are compared, it is preferably concluded that the treatment is efficacious when, for the majority of the chosen genes, and preferably for all of the chosen genes, taken individually, it can be concluded that the treatment is efficacious.

Preferentially, the comparison of the expression levels of the chosen gene(s) is carried out on a biological sample chosen from a scalp biopsy and/or a biopsy of one or more hair follicle(s), even better still a biopsy of one or more hair follicle(s) such as at least one follicular transplantation unit, in particular with a diameter of approximately 1 mm.

The treatment under consideration, evaluated by means of the method of the invention, is not limited to a particular type of treatment. It may be a treatment using one or more compounds of natural and/or synthetic origin, a natural extract, notably an essential oil, a nucleic acid, a protein complex or any other molecule or combination of molecules. It may also be a treatment using a physical means, such as waves, notably electromagnetic waves. It is preferably a topical treatment, but it is also envisioned to evaluate the efficacy of treatments administered orally, by injection, or by any other administration means.

The treatment tested can aim to reduce or inhibit hair loss related to a common alopecic state, in particular related to androgenetic alopecia, and/or to promote hair growth.

Treatments which are particularly preferred in the context of this invention are cosmetic treatments, more particularly topical cosmetic treatments.

By means of the methods of the invention, it is also possible to evaluate the efficacy of the combination of several treatments. It is in fact possible to evaluate combinations making it possible at best to restore the expression levels of one, of several or of all of the genes of the invention, such as they are expressed within a biological sample from a non-alopecic area.

By means of the methods of evaluation described above, it is possible to evaluate the efficacy of a new treatment envisioned, or else also to quantify or qualify the efficacy of already existing treatments against common alopecia, in particular androgenetic alopecia.

In this way, combinations of treatments capable of being particularly efficacious, synergistic or complementary may also be envisioned.

### Cosmetic Treatment Method

Another subject of the present invention is a method for the cosmetic treatment of a common alopecic state of the scalp, comprising at least the following steps:
a) measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2;
b) deducing from step a) whether the scalp of said subject exhibits a common alopecic state;
c) if the scalp is identified as exhibiting a common alopecic state in step b), treating said scalp with a cosmetic composition which makes it possible to induce and/or stimulate hair growth and/or to slow down hair loss,
   wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

Preferably, said at least one gene is chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes.

In another preferred embodiment, said at least one gene is chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally chosen from CTNNB1, CTNND2, MYO6, GJB6, CLDN10 and CLDN19.

In this embodiment, said at least one gene is preferably chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes and optionally the group constituted of the CTNNB1, CTNND2, MYO6, GJB6, CLDN10 and CLDN19 genes.

In another embodiment, said at least one gene is chosen from the group constituted of the CDH1, ACTB, ACTBL2, TUBB, TUBB2A, GSN, MYO3B, MYO5B, MYO6 genes and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes.

According to another embodiment, said at least one gene is chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes.

In another embodiment, said at least one gene is chosen from the group constituted of the GJB2, GJA1, GJB6 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes.

According to another embodiment, said at least one gene is chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes.

Regardless of the embodiment, said cosmetic treatment method can comprise at least one step a) of measuring the expression level of at least two, three, four or five of the abovementioned genes.

### Use of a Modulator

The present invention also relates to the use of at least one modulator of the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, or of a cosmetic composition comprising same, for the prevention and/or treatment of a common alopecic state, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

Preferably, said at least one gene is chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes.

In another preferred embodiment, said at least one gene is chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally chosen from CTNNB1, CTNND2, MYO6, GJB6, CLDN10 and CLDN19.

In this embodiment, said at least one gene is preferably chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8 genes and optionally the group constituted of the CTNNB1, CTNND2, MYO6, GJB6, CLDN10 and CLDN19 genes.

In another embodiment, said at least one gene is chosen from the group constituted of the CDH1, ACTB, ACTBL2, TUBB, TUBB2A, GSN, MYO3B, MYO5B, MYO6 genes and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes.

According to another embodiment, said at least one gene is chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes.

In another embodiment, said at least one gene is chosen from the group constituted of the GJB2, GJA1, GJB6 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes.

According to another embodiment, said at least one gene is chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes.

Regardless of the embodiment, said modulator can modulate the expression level of at least two, three, four or five of the abovementioned genes.

The term “cosmetic composition” is intended to mean a composition comprising a physiologically acceptable medium, i.e. a medium that is compatible with the skin, in particular the skin of the scalp. According to one particular embodiment, the pH of the cosmetic composition is between 4 and 7.5, notably between 4.5 and 7, and in particular between 4.7 and 6.5.

More particularly, said physiologically acceptable medium may comprise water and/or one or more water-miscible organic solvents which can be chosen from linear or branched C₁-C₆ monoalcohols such as ethanol, isopropanol, tert-butanol or n-butanol; polyols such as glycerol, propylene glycol, hexylene glycol (or 2-methyl-2,4-pentanediol), and polyethylene glycols; polyol ethers such as dipropylene glycol monomethyl ether; and mixtures thereof.

Preferably, the composition has a water content ranging from 20% to 95% by weight, even better still from 40% to 90% by weight relative to the total weight of the composition.

Advantageously, the composition comprises one or more water-miscible organic solvents in a content ranging from 0.5% to 25% by weight, preferably from 5% to 20% by weight, even better still from 10% to 15% by weight relative to the total weight of the composition.

A composition containing said modulator can preferably be administered topically.

The composition may also comprise other compounds different than the modulators of the abovementioned genes, but known for their hair loss-combating/hair regrowth activity.

The support may be of diverse nature according to the type of composition considered.

As more particularly regards compositions intended for external topical administration, they may be aqueous, aqueous-alcoholic or oily solutions, solutions or dispersions of the lotion or serum type, emulsions of liquid or semi-liquid consistency of the milk type, obtained by dispersing a fatty phase in an aqueous phase (O/W) or vice versa (W/O), or suspensions or emulsions, of soft, semi-solid or solid consistency, of the cream type, aqueous or anhydrous gel, microemulsions, microcapsules, microparticles, or vesicular dispersions of ionic and/or nonionic type.

These compositions are prepared according to the usual methods.

These compositions may notably constitute cleansing, protective, treatment or care creams, lotions, gels or mousses for caring for the scalp.

They may be used for the scalp in the form of solutions, creams, gels, emulsions or mousses, or alternatively in the form of aerosol compositions also containing a pressurized propellant.

A topical composition according to the invention may advantageously be formulated in any galenical form that is suitable for haircare, notably in the form of a hair lotion, a hair gel, a shampoo, a conditioner, a detangler, a hair cream or gel, a styling lacquer, a hairsetting lotion, a treating lotion, a dye composition (especially for oxidation dyeing) optionally in the form of a coloring shampoo, a hair-restructuring lotion, a permanent-waving composition, an antiparasitic shampoo or a medicated shampoo, notably an anti-seborrhea shampoo, a scalp care product, which is notably anti-irritant, antiaging or restructuring.

When a composition of the invention is an emulsion, the proportion of the fatty phase may range from 5% to 80% by weight and preferably from 10% to 50% by weight relative to the total weight of the composition. The oils, emulsifiers and coemulsifiers used in the composition in emulsion form are chosen from those conventionally used in cosmetics and/or dermatology. The emulsifier and the coemulsifier may be present in the composition in a proportion ranging from 0.3% to 30% by weight and preferably from 0.5% to 20% by weight relative to the total weight of the composition.

When the composition of the invention is an oily solution or gel, the fatty phase may represent more than 90% of the total weight of the composition.

In a known manner, galenical forms intended for topical administration may also contain adjuvants that are common in the cosmetic, pharmaceutical and/or dermatological field, such as hydrophilic or lipophilic gelling agents, hydrophilic or lipophilic active agents, preserving agents, antioxidants, solvents, fragrances, fillers, screening agents, odor absorbers, additional hair loss-combating or hair regrowth active agents other than the modulators of the expression of the abovementioned genes, and colorants. The amounts of these various adjuvants are those conventionally used in the field under consideration, for example from 0.01% to 20% of the total weight of the composition. Depending on their nature, these adjuvants may be introduced into the fatty phase and/or into the aqueous phase.

Method for identifying an active compound

The present invention also relates to a method for identifying a compound which allows the prevention and/or treatment of a common alopecic state, comprising the following steps:
a) bringing a compound to be tested into contact with a biological sample;
b) measuring the expression level, in said biological sample, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2;
c) selecting a compound for which the expression level of said at least one gene measured in step b) is increased compared to its expression level in the absence of said compound,
   wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

The term “increased level” is intended to mean here a level that is statistically significantly increased compared to the expression level of said at least one gene in the absence of said compound.

Preferably, said at least one gene is chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19 genes, and optionally the group constituted of the CTNNB1 and CTNND2 genes.

In another preferred embodiment, said at least one gene is chosen from CDH1, ACTB, ACTBL2, TUBB, TUBB2A, GSN, MYO3B, MYO5B, DSG2, DSG3, DSG4, DSC2, GJB2, GJA1, GJA3, TJP2, CLDN8 and optionally chosen from CTNNB1, CTNND2, MYO6, GJB6, CLDN10 and CLDN19.

In this embodiment, said at least one gene is preferably chosen from the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CL10, CLD19 genes and optionally the group constituted of the CTNNB1, CTNND2, MYO6, GJB6, CLDN10 and CLDN19 genes.

In another embodiment, said at least one gene is chosen from the group constituted of the CDH1, ACTB, ACTBL2, TUBB, TUBB2A, GSN, MYO3B, MYO5B, MYO6 genes and optionally the group constituted of the DSG2, DSG3, DSG4, DSC2, GJB2, GJA1, GJB6, GJA3, TJP2, CLDN8, CLDN10, CLDN19, CTNNB1 and CTNND2 genes.

According to another embodiment, said at least one gene is chosen from the group constituted of the DSG2, DSG3, DSG4 and DSC2 genes, and optionally the group constituted of the CDH1, ACTB, ACTBL2, TUBB, TUBB2A, GSN, MYO3B, MYO5B, MYO6, GJB2, GJA1, GJB6, GJA3, TJP2, CLDN8, CLDN10, CLDN19, CTNNB1 and CTNND2 genes.

In another embodiment, said at least one gene is chosen from the group constituted of the GJB2, GJA1, GJB6 and GJA3 genes, and optionally the group constituted of the CDH1, ACTB, ACTBL2, TUBB, TUBB2A, GSN, MYO3B, MYO5B, MYO6, DSG2, DSG3, DSG4, DSC2, TJP2, CLDN8, CLDN10, CLDN19, CTNNB1 and CTNND2 genes.

According to another embodiment, said at least one gene is chosen from the group constituted of the TJP2, CLDN8, CLDN10 and CLDN19 genes, and optionally the group constituted of the CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, CTNNB1 and CTNND2 genes.

Regardless of the embodiment, said method for identifying a compound can comprise at least one step b) of measuring the expression level of at least two, three, four or five of the abovementioned genes.

Advantageously, the biological sample is chosen from a scalp biopsy, an in vitro reconstructed scalp model, a biopsy of one or more hair follicle(s), an in vitro reconstructed hair follicle model, even better still a biopsy of one or more hair follicle(s), such as at least one follicular transplantation unit, in particular with a diameter of approximately 1 mm.

The expression “at least one” is equivalent to “one or more”.

The expressions “between ... and ...” and “ranging from ... to ...”, “at least...” or “at most” should be understood as being limits inclusive, unless otherwise specified.

The examples and figures that follow are provided as illustrations which do not limit the field of the invention.

### FIGURES

FIG. 1: Identification of the alopecic areas (top of the head) and non-alopecic areas (back of the head).

FIG. 2: Sampling of a follicular unit in an alopecic area (top of the head).

FIG. 3: Follicular unit sampled in an alopecic area (top of the head).

### EXAMPLES

### A - Characterization of a Common Alopecic State

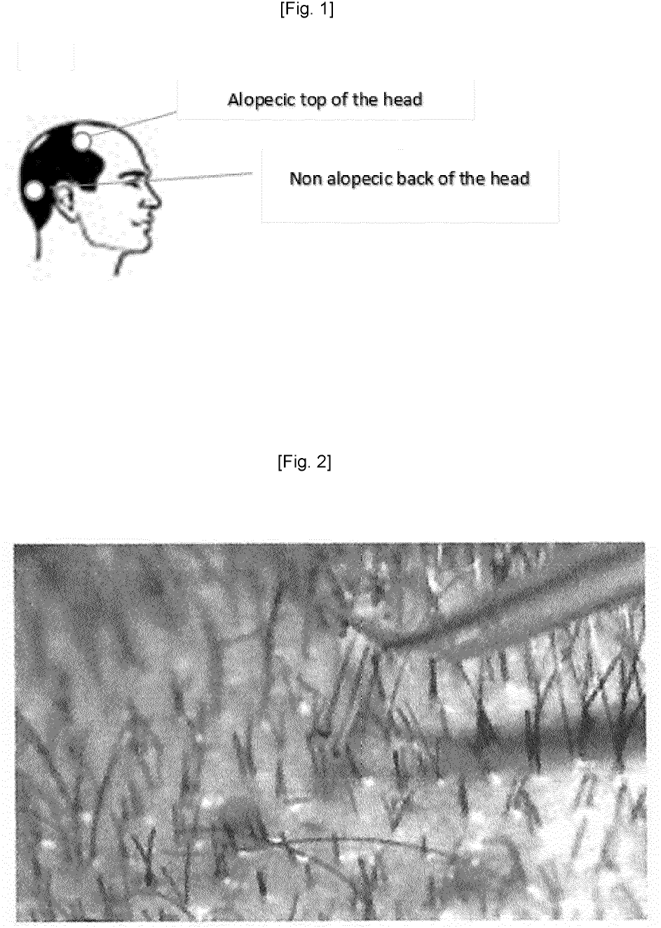

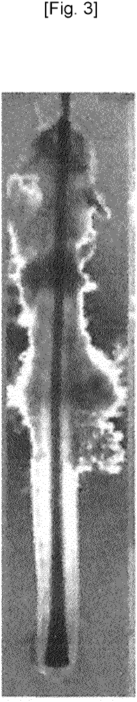

A transcriptomic study by high-throughput sequencing (RNA-sequencing) was carried out on a category of 10 volunteers suffering from grade-4 androgenetic alopecia on the Hamilton scale (age group 23-66 years old) by dissecting anagen-phase follicular units from biopsies taken on the two areas of their scalp: the alopecic area on the top of the head and the non-alopecic area at the back of the head (FIGS. 1, 2 and 3). Ten follicular units per area and per individual were isolated. A total of 20 samples, each of 10 follicular units, was thus obtained.

The total RNA was isolated from these samples with the RNeasy kit (Qiagen, Germantown, MD, United States). The purity and integrity of the RNA were evaluated on the Agilent 2100 bioanalyzer with the RNA 6000 NanoLabChip kit (Agilent, Palo Alto, California, United States) and Nanodrop spectrophotometry. All the samples had RIN values > 7.

For the preparation of the libraries and the sequencing of the RNA, 75 ng of total RNA were subjected to reverse transcription to complementary DNA (cDNA) of the first RNA strand using a mixture of random and poly-dT primers, and with the DNase treatment step being omitted. The synthesis of the second strand, using a nucleotide analog allowing its retention, made it possible to generate the double-stranded cDNA. The latter was fragmented into pieces of 200 to 500 bases. Next, a final repair was carried out to generate a blunt-ended, double-stranded cDNA, followed by ligation of the indexed adapters, a stand selection via a targeted degradation using a nucleotide analog and a reduction of the ribosomal RNA content. Finally, cDNA libraries were created by 18-cycle PCR enrichment. Equimolar amounts of each library were sequenced on a NextSeq 500, for a 75 nucleotide base ‘paired-end’ sequencing.

The results are given in the following table 2:

TABLE 2

| Genes | Gene names | adjusted p-value | Fold change (FC) | Log2-FC | % of subjects with an FC ≤ -1.25 |
|---|---|---|---|---|---|
| Adherent junctions | | | | | |
| CDH1 | cadherin 1 | 3.90×10⁻³ | -1.30 | -0.36 | 70 |
| ACTB | beta-actin | 1.18×10⁻³ | -1.28 | -0.36 | 70 |
| ACTBL2 | beta-actin-like 2 | 1.26×10⁻³ | -4.91 | -2.30 | 100 |
| TUBB | tubulin beta, class I | 1.36×10⁻² | -1.24 | -0.31 | 70 |
| TUBB2A | tubulin beta 2A, class IIa | 8.23×10⁻³ | -1.47 | -0.55 | 80 |
| GSN | gelsolin | 7.75×10³ | -1.34 | -0.42 | 80 |
| MYO3B | myosin-IIIb | 2.86×10⁻³ | -1.65 | -1.65 | 90 |
| MYO5B | myosin-Vb | 5.36×10⁻³ | -1.39 | -0.47 | 70 |
| MYO6 | myosin-VI | 6.24×10⁻³ | -1.26 | -0.33 | 70 |

Desmosomes

| | | | | | |
|---|---|---|---|---|---|
| DSG2 | desmoglein 2 | 4.89×10⁻² | -1.40 | -0.48 | 60 |
| DSG3 | desmoglein 3 | 1.56×10⁻³ | -1.37 | -0.45 | 70 |
| DSG4 | desmoglein 4 | 4.66×10⁻⁴ | -4.68 | -2.23 | 100 |
| DSC2 | desmocollin 2 | 2.98×10⁻⁵ | -1.72 | -0.78 | 100 |
| GJB2 | gap junction protein beta 2 | 7.09×10⁻⁴ | -1.86 | -0.89 | 90 |
| GJA1 | gap junction protein alpha 1 | 1.97×10⁻⁵ | -1.78 | -0.83 | 100 |
| GJB6 | gap junction protein beta 6 | 2.59×10⁻⁵ | -2.54 | -1.34 | 100 |
| GJA3 | gap junction protein alpha 3 | 6.24×10⁻⁵ | -2.86 | -1.51 | 100 |

Tight junctions

| TJP2 | tight junction protein | 1.41×10⁻³ | -1.33 | -0.41 | 100 |
|---|---|---|---|---|---|
| CLDN8 | claudin 8 | 1.66×10⁻² | -1.54 | -0.62 | 90 |
| CLDN10 | claudin 10 | 4.09×10⁻² | -1.53 | -0.62 | 70 |
| CLDN19 | claudin 19 | 2.80×10⁻³ | -3.66 | -1.87 | 90 |
| Genes which interact with CDH1 | | | | | |
| CTNNB1 | catenin beta 1 | 1.63×10⁻⁵ | -1.65 | -0.73 | 100 |
| CTNND2 | catenin delta 1 | 2.10×10⁻³ | -2.05 | -1.03 | 90 |

The results above show that at least 60% of the alopecic volunteers selected exhibit the differentiating expression criteria making it possible to demonstrate a considerable imbalance in transcripts encoding intercellular junctions which make up the hair follicle (table 2). All of these transcripts or genes are specifically overexpressed in growing hair follicles and, consequently, specifically underexpressed in alopecic hair follicles.

1. A method for the in vitro prognosis and/or diagnosis of a common alopecic state of the scalp in a subject, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

2. The method as claimed in claim 1, further comprising the following steps:
b) comparing the level of said at least one gene measured in step a) with a control;
c) on the basis of the comparison of step b), determining whether the scalp of said subject exhibits a common alopecic state.

3. The method as claimed in claim 1, wherein said scalp is confirmed as exhibiting a common alopecic state when the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, measured in step a) as is decreased compared to a control level.

4. An in vitro method for evaluating the efficacy of a treatment of a common alopecic state, comprising at least one step a) of measuring the expression level, in a biological sample from an area suspected of being an alopecic area or of becoming an alopecic area in said subject, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, before and after treatment, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

5. The method as claimed in claim 4, wherein the treatment is considered to be efficacious for the treatment of a common alopecic state when the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB1 and CTNND2, measured in step a) is increased after treatment compared to the expression level of said at least one gene before treatment.

6. A modulator of the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, for use thereof in the prevention and/or treatment of a common alopecic state, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

7. A cosmetic composition comprising one or more modulator(s) of the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, for use thereof in the prevention and/or treatment of a common alopecic state, wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

8. A method for identifying a compound which allows the prevention and/or treatment of a common alopecic state, comprising the following steps:
a) bringing a compound to be tested into contact with a biological sample;
b) measuring the expression level, in said biological sample, of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2;
c) selecting a compound for which the expression level of said at least one gene measured in step b) is increased compared to its expression level in the absence of said compound,
wherein said common alopecic state is chosen from androgenetic alopecia, traction alopecia, female pattern hair loss, cicatricial alopecia, telogen effluvium, stress-related alopecia, seasonal alopecia, age-related alopecia and micro-inflammatory alopecia.

9. The method as claimed in claim 8, wherein the biological sample is chosen from a scalp biopsy, an in vitro reconstructed scalp model, a biopsy of one or more hair follicle(s), and an in vitro reconstructed hair follicle model.

10. The method as claimed in claim 2, wherein said scalp is confirmed as exhibiting a common alopecic state when the expression level of at least one gene involved in the intercellular junctions of the scalp and/or of the hair follicle chosen from CDH1, ACTB, ACTBL2, TUBB, TUBB2A, GSN, MYO3B, MYO5B, MYO6, DSG2, DSG3, DSG4, DSC2, GJB2, GJA1, GJB6, GJA3, TJP2, CLDN8, CLDN10, CLDN19, and optionally of at least one gene chosen from CTNNB 1 and CTNND2, measured in step a) as is decreased compared to a control level.
